(19) United States
(12) Patent Application Publication
(10) Pub. No.: US 2025/0195246 A1
(43) Pub. Date: Jun. 19, 2025

(54) ENDOVASCULAR BRANCH COUPLER (EBC) STENT GRAFT

(71) Applicant: University of Southern California, Los Angeles, CA (US)

(72) Inventor: Sukgu Michael HAN, South Pasadena, CA (US)

(73) Assignee: University of Southern California, Los Angeles, CA (US)

(21) Appl. No.: 18/985,634

(22) Filed: Dec. 18, 2024

Related U.S. Application Data
(60) Provisional application No. 63/611,949, filed on Dec. 19, 2023.

(51) Int. Cl. A61F 2/95

(52) U.S. Cl. CPC A61F 2/95

(57) ABSTRACT

An endovascular branch coupler stent graft includes a tubular stent assembly defining a central opening and four branch channels disposed in the central opening and attached to the tubular stent assembly. The four branch channel extends along the length of tubular stent assembly. Each branch channel is configured to receive a covered stent for catheterizing a branch vessel. Similarly, the central opening configured to receive a primary stent graft for reinforcing weakened sections of a subject's aorta.

### CROSS-REFERENCE TO RELATED APPLICATIONS

This application claims the benefit of U.S. provisional application Ser. No. 63/611,949 filed Dec. 19, 2023, the disclosure of which is hereby incorporated in its entirety by reference herein.

### TECHNICAL FIELD

In at least one aspect, the present invention is related to endovascular stents.

### BACKGROUND

The majority of abdominal aortic aneurysms (AAAs) are now repaired using endovascular stent grafts delivered through percutaneous punctures of the femoral arteries. This minimally invasive approach offers several significant advantages over traditional open aortic repairs, including a markedly lower risk of major perioperative complications and a considerably faster postoperative recovery time. These benefits have made endovascular techniques the preferred method for treating most AAAs.

However, approximately 20-30% of AAAs involve segments of the aorta that include branch arteries supplying critical organs such as the kidneys, liver, and intestines. These complex cases are classified as complex AAAs (cAAAs) or thoracoabdominal aortic aneurysms (TAAAs). Treating such aneurysms poses unique challenges due to the involvement of these vital branch vessels. Currently, the market offers only limited solutions for managing cAAAs and TAAAs. The sole commercially available device designed for this purpose is the Zenith Fenestrated stent graft (Cook Medical, Bloomington, IN), which can accommodate up to three branch vessels. This device achieves this by using custom-manufactured fenestrations or holes that allow branch stents to be inserted into the branch vessels.

While effective in some cases, the Zenith Fenestrated stent graft has notable limitations. The number of fenestrations is restricted, limiting its applicability for aneurysms involving more than three branch vessels. Moreover, the process of custom manufacturing and shipping the device can be time-intensive, delaying critical treatment for patients in need. These drawbacks highlight the unmet need for more versatile and readily available stent graft solutions for the management of complex aortic aneurysms.

Accordingly, there is a need for improved methods for treating aortic aneurysms.

### SUMMARY

In at least one aspect, an endovascular branch coupler stent graft is provided. The endovascular branch coupler stent graft includes a tubular stent assembly defining a central opening and four branch channels disposed in the central opening and attached to the tubular stent assembly. Each of the four branch channel extend along the length of tubular stent assembly. Each branch channel is configured to receive a covered stent for catheterizing a branch vessel. Characteristically, the central opening configured to receive a primary stent graft for reinforcing weakened sections of a subject's aorta.

In another aspect, a method for deploying the endovascular branch coupler stent graft described herein is provided. The method includes a step of loading the endovascular branch coupler stent graft into a dedicated sheath with preloaded wires through each of the four branch channels to facilitate catheterization of the four branch channels. The endovascular branch coupler stent graft is delivered to a treatment zone. The endovascular branch coupler stent graft is deployed proximal to target branch vessels using a pin-and-pull maneuver. The method also includes a step of sequentially catheterizing and stenting the branch vessel. The primary stent graft is deployed for reinforcing weakened sections of the subject's aorta into endovascular branch coupler stent graft.

Advantageously, the Endovascular Branch Coupler (EBC) Stent Graft provides a transformative solution to several limitations of prior art, offering significant advantages in endovascular repair procedures. Unlike custom-manufactured alternatives, the EBC is an off-the-shelf stent graft, eliminating the delays and expenses associated with bespoke fabrication. Its innovative design accommodates up to four branch vessels, enabling seamless incorporation during the repair of complex abdominal aortic aneurysms (AAA) and thoracoabdominal aortic aneurysms (TAAA). Furthermore, the EBC is engineered for compatibility with existing commercial devices, ensuring integration without the need for specialized equipment or modifications. Additionally, the EBC extends its utility to the aortic arch, facilitating endovascular repairs that incorporate arch branch vessels, thus addressing a critical challenge in advanced aortic interventions. This comprehensive approach enhances procedural efficiency, reduces patient risk, and expands the scope of endovascular treatment options.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements and wherein:

FIG. 1A. Perspective side view of an Endovascular Branch Coupler.

FIG. 1B. Perspective side view of an Endovascular Branch Coupler.

FIG. 1C. Top view of an Endovascular Branch Coupler.

FIGS. 2A and 2B. Schematic depicting application of the Endovascular Branch Coupler (EBC) Stent Graft for the treatment of (A) complex abdominal aortic aneurysms (cAAA) and (B) thoracoabdominal aortic aneurysms (TAAA).

FIG. 3. Schematic showing application of the Endovascular Branch Coupler (EBC) Stent Graft for the treatment of aortic arch aneurysms.

FIGS. 4A and 4B. Schematic flowchart depicting the deployment steps of the Endovascular Branch Coupler (EBC) Stent Graft. The EBC is preloaded into a dedicated sheath, with guidewires pre-positioned through each of the four branch channels (Step A). Using a femoral artery approach, the EBC is advanced to the desired location and deployed proximal to the target branch vessels through a precise pin-and-pull maneuver (Steps B and C). Once deployed, each branch vessel is catheterized sequentially and reinforced with covered stents to ensure proper integration and blood flow (Step D). The procedure is finalized by completing the endovascular aortic repair with the deployment of standard thoracic endovascular aortic repair (TEVAR) or endovascular aneurysm repair (EVAR) devices into the EBC framework (Step E).

FIGS. 5A, 5B, 5C and 5D. First human implant of EBC in an urgent endovascular repair of TAAA. EBC prototype was constructed by modifying the Alpha Thoracic stent graft with 4 Viabahan covered stents (A, B). 3-D reconstruction of postoperative CT scan (C). Completion angiography showing EBC (red), successfully implanted along with commercially available branch stents (green), and TEVAR and EVAR devices (blue) (D).

### DETAILED DESCRIPTION

Reference will now be made in detail to presently preferred compositions, embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. Also, unless expressly stated to the contrary: percent, “parts of,” and ratio values are by weight; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

The phrase “composed of” means “including” or “comprising.” Typically, this phrase is used to denote that an object is formed from a material.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4 . . . 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits.

Throughout this application, where publications are referenced, the disclosures of these publications in their entireties are hereby incorporated by reference into this application to more fully describe the state of the art to which this invention pertains.

### ABBREVIATIONS

“AAA” means abdominal aortic aneurysms.
“CAAA” means complex abdominal aortic aneurysms.
“EBC” means endovascular branch coupler.
“ePTFE” means expanded polytetrafluoroethylene.
“EVAR” means endovascular aneurysm repair.
“TEVAR” means thoracic endovascular aneurysm repair.
“TAAA” means thoracoabdominal aortic aneurysms.

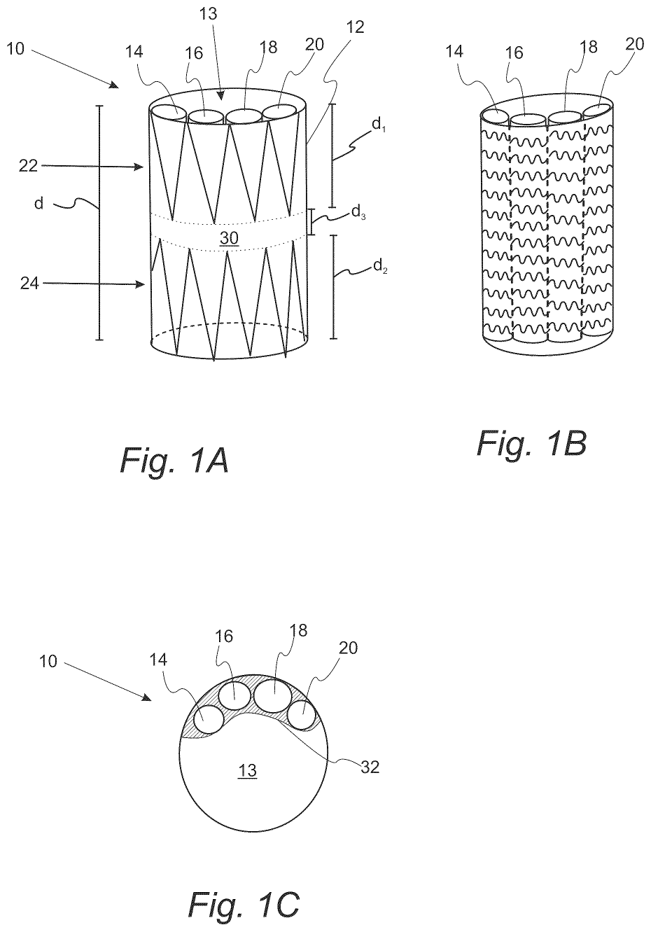

Referring to FIGS. 1A, 1B, and IC, detailed schematics of the Endovascular Branch Coupler (EBC) Stent Graft 10 are provided. The EBC Stent Graft 10 comprises a tubular stent assembly 12 that defines a central opening 13. In a refinement, the tubular stent assembly 12 can be constructed from high-strength stainless steel or Nitinol stents, which are coupled with durable polyester or expanded polytetrafluoroethylene fabric, ensuring both flexibility and structural integrity. Nitinol is a metal alloy composed of nickel (Ni) and titanium (Ti), typically in nearly equal proportions. Nitinol is known for its unique shape memory effect and superelasticity, allowing it to return to its original shape after deformation when exposed to heat or upon removal of stress. It is highly biocompatible and corrosion-resistant, making it ideal for medical applications such as stents, orthodontic wires, and surgical instruments. In a refinement, EBC Stent Graft 10 can be held in place via a radial force generated by expansion.

Strategically positioned within the central opening are four branch channels, designated as 14, 16, 18, and 20, which are securely attached to the tubular stent assembly 12. These branch channels 14, 16, 18, and 20 extend along the full length of the tubular stent section 12, ensuring uniform support and precise alignment. In a refinement, the branch channels 14, 16, 18, and 20 are arranged to be substantially parallel, optimizing spatial configuration and functionality. Each branch channel is specifically designed to accommodate a covered stent, enabling efficient catheterization of branch vessels during deployment. In a refinement, each of the four branch channels are reinforced with nitinol stents and fabric.

Furthermore, the central opening of the tubular stent assembly 12 is engineered to receive a primary stent graft, such as a thoracic endovascular aortic repair (TEVAR) or endovascular aneurysm repair (EVAR) device, for reinforcing weakened sections of a subject's aorta. This modular design enhances the versatility of the EBC Stent Graft, making it adaptable to various complex anatomical scenarios.

In another aspect, the Endovascular Branch Coupler (EBC) Stent Graft 10 is characterized by a length d ranging from 4 to 6 cm, with a preferred length of approximately 5 cm in one refinement. The tubular stent assembly 12 is further refined to include two rows of Z stents: a first Z stent row 22 and a second Z stent row 24. Z stents are vascular stents with a zigzag structural design, providing flexibility and radial strength for various medical applications. Therefore, these rows provide structural support and stability for the stent graft. The first Z stent row 22 and the second Z stent row 24 are designed with first predetermined length d₁ and second predetermined length d₂, respectively. Typically, both d₁ and d₂ independently range from about 18 mm to 25 mm, with an optimized length of approximately 22 mm in one refinement. To ensure proper separation and flexibility between the rows, a fabric section 30 is interposed, defining a third predetermined length d₃. Therefore, the first predetermined length d₁ and the second predetermined length d₂ are separated by fabric section of a third predetermined length d₃. This fabric section 30 enhances the device's adaptability and overall performance.

In another aspect, the length d₃ of the fabric section 30 is between 3 mm and 8 mm, with a preferred length of approximately 6 mm. These precise dimensional specifications contribute to the EBC Stent Graft's robust design, enabling reliable deployment and optimal integration with vascular anatomy.

In another aspect, the diameter of the endovascular branch coupler stent graft 10 can be from about 30 mm to 40 mm. In a refinement, the endovascular branch coupler stent graft 10 has a diameter of about 34 mm or 38 mm. Similarly, each of the four branch channels 14, 16, 18, and 20 has a diameter from 6 to 10 mm and optimally about 8 mm.

In another aspect, each of the four branch channels 14, 16, 18, and 20 is reinforced with the nitinol stents and fabric. Moreover, gap spaces between each of the four branch channels 14, 16, 18, and 20 are covered with fabric 32. EBC will be loaded in a sheath with a preloaded wire mechanism for delivery and deployment of the stent from any suitable vascular access sites.

In another aspect, the Endovascular Branch Coupler (EBC) Stent Graft may include one or more additional branch channels, enhancing its versatility and adaptability for complex vascular anatomies. These additional branch channels can be strategically integrated into the stent graft to accommodate more extensive branching arteries, such as those supplying critical organs or regions with high anatomical variability. This configuration allows the EBC Stent Graft to address a broader range of clinical scenarios, including aneurysms involving multiple visceral or peripheral branch vessels beyond the standard four-channel design. The additional branch channels may be reinforced with materials such as Nitinol and ePTFE to maintain structural integrity and prevent complications such as kinking or collapse during deployment. Furthermore, the inclusion of these channels can facilitate the incorporation of advanced covered stents, providing robust and reliable support to the extended vascular network. This expanded capability makes the EBC Stent Graft a highly flexible solution for endovascular repair, catering to both routine and complex cases with equal efficacy.

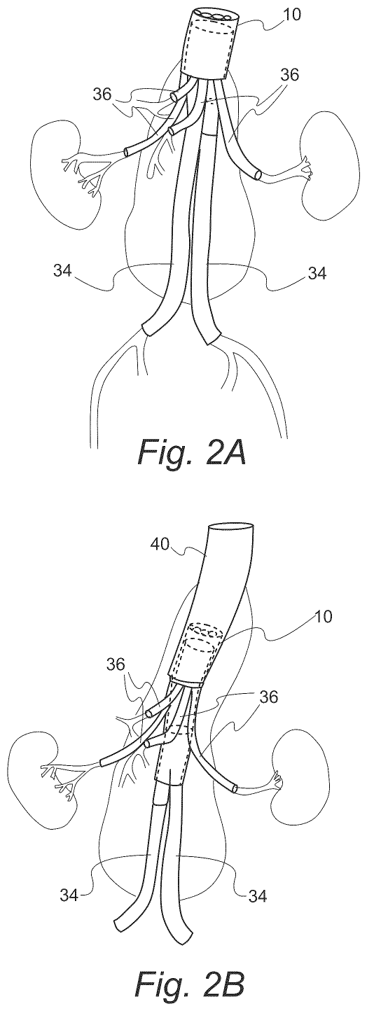

Advantageously, the Endovascular Branch Coupler (EBC) Stent Graft is designed as an off-the-shelf solution, eliminating the need for custom manufacturing and the associated delays, while providing unparalleled versatility for complex aortic repairs. This innovative stent graft enables the seamless incorporation of visceral and renal arteries during endovascular procedures by effectively linking commercially available thoracic and abdominal bifurcated endovascular aortic stent grafts with covered stents. The modular design ensures compatibility with existing devices, allowing clinicians to customize the treatment approach based on individual patient anatomy and clinical requirements. This flexibility permits the treatment of a wide range of complex abdominal aortic aneurysms (cAAAs) and thoracoabdominal aortic aneurysms (TAAAs), as depicted in FIGS. 2A and 2B. FIG. 2A depicts the application of endovascular branch coupler stent graft 10 for the treatment of complex abdominal aortic aneurysms. In this variation, endovascular branch coupler stent graft 10 receives EVAR device 34 in its central cavity. Similarly, FIG. 2B depicts a variation for the treatment of thoracoabdominal aortic aneurysms. In this variation, endovascular branch coupler stent graft 10 receives EVAR device 34 in its central cavity, while TEVAR device 38 is positioned over endovascular branch coupler stent graft 10. In each variation, individual branch vessels are stented with covered stents 36. Covered stents 36 can be held in place in the coupler stent graft 10 and branch arteries via a radial force generated by expansion. By accommodating multiple configurations, the EBC Stent Graft offers tailored solutions for complex vascular anatomies involving branch arteries supplying vital organs such as the kidneys, liver, and intestines. This capability addresses a critical gap in current endovascular technology, enabling the treatment of aneurysms with greater efficiency, reduced risk, and expanded procedural applicability. The EBC's off-the-shelf availability ensures timely intervention for patients, making it a transformative tool in the management of challenging vascular conditions.

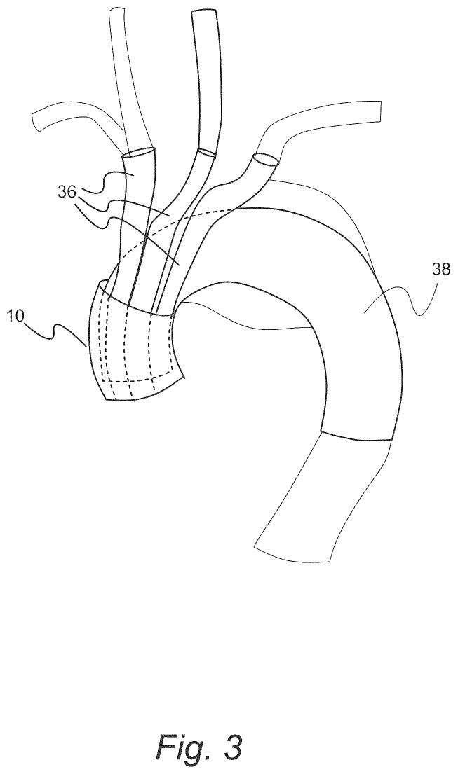

Moreover, the Endovascular Branch Coupler (EBC) Stent Graft extends its utility to the treatment of aortic arch aneurysms and dissections, offering a streamlined, off-the-shelf solution that eliminates the need for custom devices. This innovative design enables the incorporation of arch branch arteries by effectively linking commercially available thoracic endovascular aortic stent grafts with covered stents. This capability allows for precise and reliable repair of complex aneurysms and dissections in the aortic arch, as illustrated in FIG. 3. In this variation, endovascular branch coupler stent graft 10 receives primary stent device 38 in its central cavity. As set forth above, individual branch vessels are stented with covered stents 36. By providing a modular and adaptable system, the EBC Stent Graft facilitates endovascular repair in one of the most anatomically challenging regions of the vasculature. It ensures compatibility with existing devices while delivering the flexibility needed to address the unique branching patterns of the aortic arch. This approach significantly simplifies procedural planning and execution, reduces operative risk, and enhances outcomes by maintaining blood flow to critical branch vessels. The EBC's application in aortic arch repairs represents a significant advancement in the treatment of life-threatening vascular conditions, improving both accessibility and effectiveness of care.

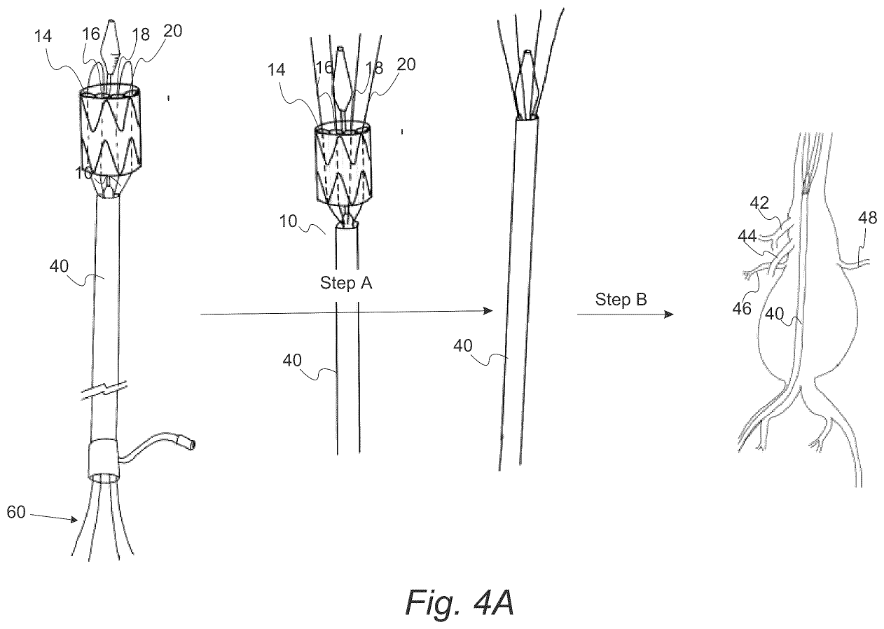

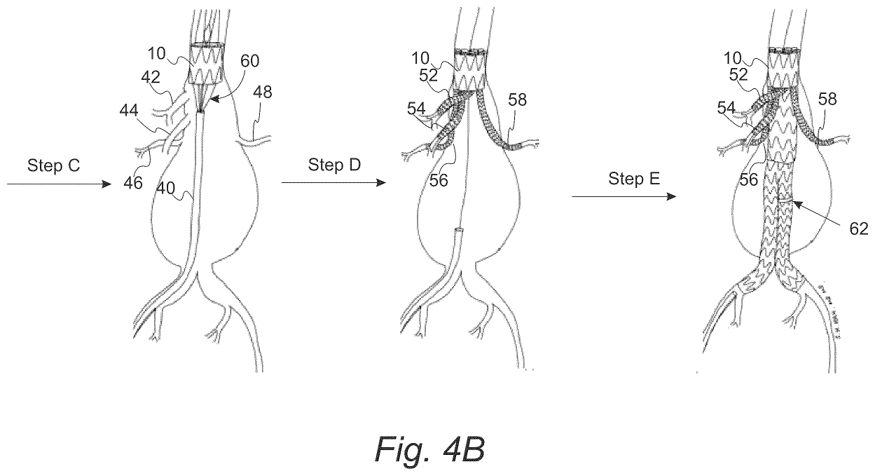

FIG. 4 depicts a method for deploying the endovascular branch coupler stent graft of FIG. 1. In step A), endovascular branch coupler stent graft 10 is loaded into a dedicated sheath 40 with preloaded wires through each of the four branch channels 14, 16, 18, and 20 to facilitate catheterization of each of the four branch channels. In steps B), endovascular branch coupler stent graft 10 is delivered to the treatment zone. FIG. 2 depicts a variation where endovascular branch coupler stent graft 10 is delivered to the treatment zone through femoral arterial access over a guidewire. In step C), endovascular branch coupler stent graft 10 is deployed proximal to the target branch vessels 42, 44, 46, 48 using a pin-and-pull maneuver. In step D), branch vessels are then catheterized sequentially and stented using covered stents 52, 54, 56, and 58 which can be introduced along guide wires 60. In step E), endovascular aortic repair is completed by deployment of a primary stent graft 60 for reinforcing weakened sections of a subject's aorta (e.g., a standard TEVAR device or EVAR device) into endovascular branch coupler stent graft 10. In a variation, the method can be deployed treat complex abdominal aortic aneurysms or thoracoabdominal aortic aneurysms. In another variation, the method can be deployed to treat aortic arch aneurysm.

The following examples illustrate the various embodiments of the present invention. Those skilled in the art will recognize many variations that are within the spirit of the present invention and scope of the claims.

### Synopsis of First in Human Implant

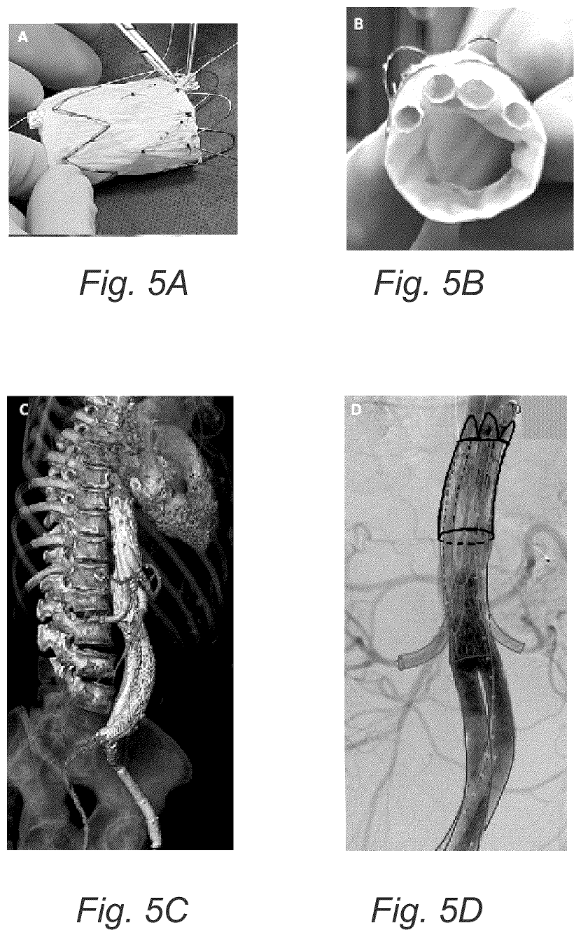

A prototype of EBC was successfully utilized in a 77 year old patient with symptomatic extent IV thoracoabdominal aortic aneurysm. The patient was successfully treated with total endovascular repair using the EBC concept. After a full disclosure of the off-label nature of this procedure to the patient and her family, an urgent endovascular repair was performed. The EBC prototype was constructed by modifying a standard commercially available 28 mm×109 mm Zenith Alpha Thoracic stent grafts (Cook Medical, Bloomington, IN) as the aortic component of EBC, and using 8 mm×50 mm Viabahn covered stents (W.L. Gore & Associates, Flagstaff, AZ) (FIG. 5A, B). The steps outlined above were followed. Viabahn stents were deployed to bridge the visceral and renal target arteries with the corresponding branch channels. Conformable TAG and Excluder (W.L. Gore & Associates, Flagstaff, AZ) were deployed below the EBC to complete the aneurysm repair (FIG. 5C, D). Conformable TAG and Excluder are commercially available endovascular devices for treating aortic aneurysms and related vascular conditions. Implants were performed successfully, and the patient recovered well without complications. A Type III endoleak was repaired using an additional stent graft into the Excluder. This successful case serves as a proof of concept and demonstrates the feasibility of the EBC. Further refinement of the procedure is expected with dedicated device manufacturing.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. An endovascular branch coupler stent graft comprising:
a tubular stent assembly defining a central opening and a length; and
four branch channels disposed in the central opening and attached to the tubular stent assembly, the four branch channel extending along the length of tubular stent assembly, each branch channel being configured to receive a covered stent for catheterizing a branch vessel, the central opening configured to receive a primary stent graft for reinforcing weakened sections of a subject's aorta.

2. The endovascular branch coupler stent graft of claim 1, wherein the primary stent graft is a thoracic endovascular aneurysm repair device or an endovascular aneurysm repair device.

3. The endovascular branch coupler stent graft of claim 1, wherein the four branch channels are substantially parallel.

4. The endovascular branch coupler stent graft of claim 1, wherein the tubular stent assembly includes stainless steel or Nitinol stents coupled with durable polyester or expanded polytetrafluoroethylene fabric.

5. The endovascular branch coupler stent graft of claim 1, wherein the length is from 4 to 6 cm.

6. The endovascular branch coupler stent graft of claim 1, wherein the length of 5 cm.

7. The endovascular branch coupler stent graft of claim 1, wherein the length from 4 to 6 cm.

8. The endovascular branch coupler stent graft of claim 1, wherein the tubular stent assembly includes a first Z stent row and a second Z stent row.

9. The endovascular branch coupler stent graft of claim 8, wherein the first Z stent row has a first predetermined length and the second Z stent row has a second predetermined length.

10. The endovascular branch coupler stent graft of claim 9, wherein the first predetermined length and the second predetermined length are from about 18 mm to 25 mm.

11. The endovascular branch coupler stent graft of claim 9, wherein the first predetermined length and the second predetermined length are each independently from about 18 mm to 25 mm about 22 mm.

12. The endovascular branch coupler stent graft of claim 9, wherein the first predetermined length and the second predetermined length are separated by fabric section of a third predetermined length.

13. The endovascular branch coupler stent graft of claim 12, wherein the third predetermined length is about 6 mm.

14. The endovascular branch coupler stent graft of claim 1, wherein a diameter of the endovascular branch coupler stent graft is from about 30 mm to 40 mm.

15. The endovascular branch coupler stent graft of claim 14, wherein the diameter of about 34 mm or 38 mm.

16. The endovascular branch coupler stent graft of claim 1, wherein each of the four branch channels have a diameter from 6 to 10 mm.

17. The endovascular branch coupler stent graft of claim 1, wherein each of the four branch channels independently have a diameter of about 8 mm.

18. The endovascular branch coupler stent graft of claim 1, wherein each of the four branch channels are reinforced with nitinol stents and fabric.

19. The endovascular branch coupler stent graft of claim 1, wherein gap spaces between the four branch channels are covered with fabric.

20. The endovascular branch coupler stent graft of claim 1, further comprising one or more additional branch channels.

21. A method for deploying an endovascular branch coupler stent graft comprising:
a tubular stent assembly defining a central opening and a length; and
four branch channels disposed in the central opening and attached to the tubular stent assembly, the four branch channel extending along the length of tubular stent assembly, each branch channel being configured to receive a covered stent for catheterizing a branch vessel, the central opening configured to receive a primary stent graft for reinforcing weakened sections of a subject's aorta, the method comprising:
loading the endovascular branch coupler stent graft into a dedicated sheath with preloaded wires through each of the four branch channels to facilitate catheterization of the four branch channels;
delivering the endovascular branch coupler stent graft to a treatment zone;
deploying the endovascular branch coupler stent graft proximal to target branch vessels using a pin-and-pull maneuver;
sequentially catheterizing and stenting the branch vessel; and
deploying the primary stent graft for reinforcing weakened sections of the subject's aorta into endovascular branch coupler stent graft.

22. The method of claim 21, wherein the endovascular branch coupler stent graft is deployed to the treatment zone through femoral arterial access over a guidewire.

23. The method of claim 22, wherein the primary stent graft is an endovascular aneurysm repair device.

24. The method of claim 21, wherein the primary stent graft is a thoracic endovascular aneurysm repair device.

25. The method of claim 21, deployed to treat complex abdominal aortic aneurysms or thoracoabdominal aortic aneurysms.

26. The method of claim 21, deployed to treat aortic arch aneurysm.
